(19) United States
(12) Patent Application Publication
(10) Pub. No.: US 2023/0129540 A1
(43) Pub. Date: Apr. 27, 2023

(54) CELL ANALYZER DEVICE

(71) Applicant: SHIMADZU CORPORATION, Kyoto-shi, Kyoto (JP)

(72) Inventors: Shuhei YAMAMOTO, Kyoto-shi (JP); Ryuji SAWADA, Kyoto-shi (JP)

(73) Assignee: SHIMADZU CORPORATION, Kyoto-shi, Kyoto (JP)

(21) Appl. No.: 17/913,997

(22) PCT Filed: Feb. 3, 2021
(86) PCT No.: PCT/JP2021/003945

§ 371 Date: Nov. 9, 2022

(30) Foreign Application Priority Data
Mar. 31, 2020 (JP) 2020-063426

(51) Int. Cl. C12M 1/34; G06T 7/00

(52) U.S. Cl. CPC C12M 41/36; G06T 7/0012; G06T 2207/30024; G06T 2207/10056; G06T 2207/30072

(57) ABSTRACT

Provided is a cell analysis device including: a display unit; a storage unit; an analysis result input receiving unit to receive inputs of culture conditions and measured values of items obtained by analyzing a cell cultured under each condition, and store them in the storage unit associating the measured values with a corresponding one culture condition; a culture condition designation input receiving unit to receive input of designation of the culture condition; a correlation evaluation unit to read out measured values of items on the cell cultured under the designated culture condition from the storage unit, and evaluate a magnitude of correlation between the measured values of two items for all combinations of two of the items; and a correlation item presentation unit to display, on the display unit, a predetermined number of combinations extracted from among all the combinations based on an evaluation result by the correlation evaluation unit.

### TECHNICAL FIELD

The present invention relates to a cell analysis device.

### BACKGROUND ART

In the field of regenerative medicine, in recent years, studies using pluripotent stem cells such as iPS cells, ES cells, and pluripotent stem cells have been conducted. In research and development of regenerative medicine using such pluripotent stem cells, it is necessary to culture a large number of cells. Thus, cells are cultured under various conditions set with different values of culture parameters such as the type of culture medium and culture temperature, and a state of the obtained cells is confirmed for various items (the number of cells, the shape of cell, cell viability, differentiation state of cell, etc.) to search for an optimal culture condition. Various methods are used to confirm the state of the cell depending on a target item.

When the number and shape of cells are confirmed, cultured cells are photographed with a phase contrast microscope to acquire a cell image, and the cell image is analyzed with image processing software (for example, Non Patent Literature 1). In addition, in order to confirm a survival rate of a cell, for example, a method called trypan blue staining method is used (for example, Patent Literature 1). In the trypan blue staining method, a trypan blue solution is added to a suspension in which cells peeled from the culture medium are dispersed in a predetermined solution to prepare a sample solution. The trypan blue solution permeates only a cell membrane of dead cells and stains cytoplasm in blue. The sample solution thus obtained is fed into a cell counter, and living cells and dead cells are counted based on the difference in staining state. Further, when the differentiation state of a cell is confirmed, a method using immunostaining (for example, Patent Literature 2) or a method of quantifying an expression level of a marker gene (for example, Patent Literature 3) is used. Hereinafter, a numerical value acquired for each item for confirming the cell state is referred to as measured value.

As described above, measured values of a plurality of items related to the cell state are obtained for each culture (or for each well used for culture). When the measured values of a plurality of items obtained for each culture are analyzed, there may be correlation between the measured values of specific two items. For example, sometimes there is a correlation that a proportion of differentiated cells increases as the shape of cells becomes longer. In that case, it is possible to estimate the differentiation state of the cell from the shape of the cell, and, generally, it becomes possible to estimate a value that could have been obtained only by an invasive method from a measured value regarding the shape of the cell (for example, aspect ratio of the shape of the cell) obtained by a non-invasive method. In addition, it is possible to acquire two analysis values simply by performing one analysis, so that the efficiency of work related to confirmation of the cell state is improved.

### CITATION LIST

### Patent Literature

Patent Literature 1: JP 2005-511023 A

Patent Literature 2: JP 2004-313184 A

Patent Literature 3: JP 2006-042663 A

### Non Patent Literature

Non Patent Literature 1: Kota Miura and Yuki Tsukada, “Starting biological image analysis with ImageJ”, Gakken Medical Shujunsha Co., Ltd., April 2016, ISBN: 9784780909364

### SUMMARY OF INVENTION

### Technical Problem

When the culture condition is searched, it is often the case that a plurality of culture conditions having slightly different values of culture parameters are comprehensively set, and a plurality of cultures are performed under each condition. Then, measured values of a large number of items are acquired for each of the plurality of cultures. As a result, the total number of measured values becomes enormous. Thus, in the prior art, in order to confirm if the measured values of any two of a plurality of items have a correlation, it was necessary for an analyst to manually check the measured values of the respective items for all combinations of two of the plurality of items, which takes time and effort.

A problem to be solved by the present invention is to provide a cell analysis device capable of easily confirming correlation between measured values of a plurality of items regarding a state of a cell cultured under a plurality of same or different conditions.

### Solution to Problem

A cell analysis device according to the present invention made to solve the above problems includes:

a display unit;

a storage unit;

an analysis result input receiving unit configured to receive inputs of a plurality of culture conditions and measured values of a plurality of items obtained by analyzing a cell cultured under each of the plurality of culture conditions with a predetermined analyzer, and store the measured values in the storage unit associating the measured values of the plurality of items with a corresponding one of the plurality of culture conditions;

a culture condition designation input receiving unit configured to receive input of designation of some or all of the plurality of culture conditions;

a correlation evaluation unit configured to read out measured values of a plurality of items on the cell cultured under the designated culture condition from the storage unit, and evaluate a magnitude of correlation between the measured values of two items by a predetermined method for each of all combinations of two of the plurality of items; and

a correlation item presentation unit configured to display, on the display unit, a predetermined number of combinations extracted from among all the combinations based on an evaluation result by the correlation evaluation unit.

### Advantageous Effects of Invention

In the cell analysis device according to the present invention, when a user inputs the measured values of the plurality of items obtained by analyzing a cell cultured under each of the plurality of culture conditions with a predetermined analyzer together with the culture conditions of the cells, the analysis result input receiving unit associates the measured values with a corresponding one of the plurality of the culture conditions and stores them in the storage unit. Thereafter, when the user makes input of designation of some or all of the plurality of culture conditions, the correlation evaluation unit reads out the measured values of the plurality of items on the cell cultured under the corresponding condition from the storage unit. Then, the magnitude of the correlation is evaluated for all combinations of two of the plurality of items by a predetermined method. The predetermined method is, for example, a method in which the correlation is evaluated larger as the difference between the measured values and an approximate straight line using a linear function or an approximate curve using a quadratic function is smaller. When the evaluation result for all combinations of items is obtained, the correlation item presentation unit extracts a predetermined number of combinations based on the evaluation result and displays the extracted combinations on the display unit. In the cell analysis device according to the present invention, the correlation between the measured values of two items can be easily observed only by looking at items displayed on the display unit.

### BRIEF DESCRIPTION OF DRAWINGS

FIG. 1 is a configuration diagram of a main part of a cell analysis system in which an embodiment of a cell analysis device according to the present invention and a microscopic observation unit are combined.

FIG. 2 is a screen example for designating a culture condition to be analyzed in the cell analysis system of the present embodiment.

FIG. 3 is a diagram for explaining a correlation of a measured value in the cell analysis system of the present embodiment.

FIG. 4 is a screen example in which a combination of items having a high correlation of the measured value is displayed together with a scatter diagram for each culture condition in the cell analysis system of the present embodiment.

### DESCRIPTION OF EMBODIMENTS

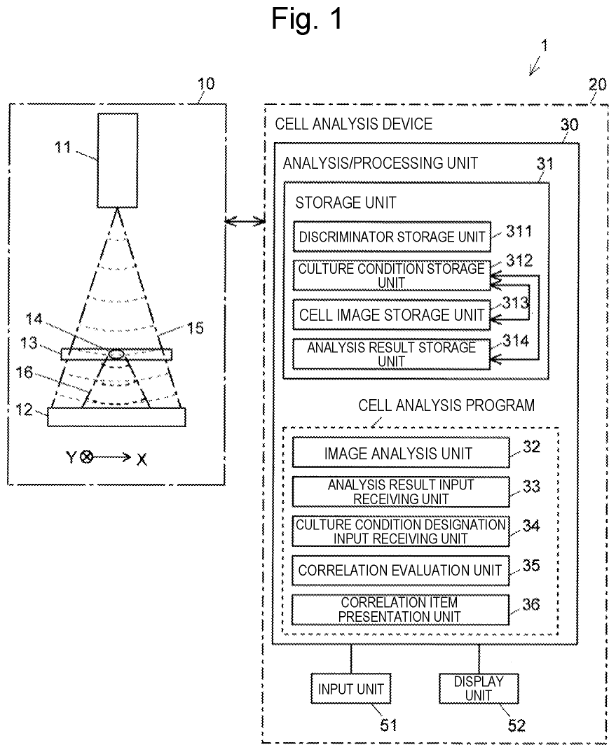

One embodiment of a cell analysis device according to the present invention will be described below with reference to the drawings. FIG. 1 is a configuration diagram of a main part of a cell analysis system 1 including a cell analysis device 20 of the present embodiment.

The cell analysis system 1 of the present embodiment is roughly composed of a microscopic observation unit 10 and the cell analysis device 20. The microscopic observation unit 10 of the present embodiment is an in-line holographic microscope (IHM) and includes a light source unit 11 including a laser diode and the like and an image sensor 12, and can acquire a phase image of a cell 14 in a culture plate 13.

The light source unit 11 irradiates a predetermined region of the culture plate 13 with coherent light having a spread at an angle of about 10°. The coherent light (object light 16) transmitted through the culture plate 13 and the cell 14 reaches the image sensor 12 while interfering with light (reference light 15) transmitted through a region close to the cell 14 on the culture plate 13. The object light 16 is light whose phase has changed when transmitted through the cell 14. On the other hand, the reference light 15 is not transmitted through the cell 14 and therefore is light without phase change caused by the cell 14. On the detection surface (image surface) of the image sensor 12, an image (hologram) by interference fringes between the object light 16 with its phase changed by the cell 14 and the reference light 15 with its phase unchanged is formed.

The light source unit 11 and the image sensor 12 are sequentially moved in an X-axis direction and a Y-axis direction in conjunction with each other by a moving mechanism (not shown). As a result, an irradiation region (observation region) of the coherent light emitted from the light source unit 11 is moved on the culture plate 13, so that it is possible to acquire hologram data (two-dimensional light intensity distribution data of the hologram formed on the detection surface of the image sensor 12) over the entire region of interest (target region for image analysis) in the culture plate.

The cell analysis device 20 includes an analysis/processing unit 30, and an input unit 51 and a display unit 52 which are connected to the analysis/processing unit 30. In addition to the storage unit 31, the analysis/processing unit 30 includes, as functional blocks, an image analysis unit 32, an analysis result input receiving unit 33, a culture condition designation input receiving unit 34, a correlation evaluation unit 35, and a correlation item presentation unit 36. The analysis/processing unit 30 may be constituted of a personal computer and a workstation, and the above-described functional blocks are embodied by executing a cell analysis program installed in advance.

The storage unit 31 is provided with a discriminator storage unit 311. The discriminator storage unit 311 stores a plurality of different discriminators depending on the type of the cell to be analyzed and the analysis content. The type of the cell is, for example, an iPS cell, an ES cell, or a cancer cell. The analysis content is, for example, determination of the number of cells, determination of the shape of the cell, determination of cell coverage, and determination of a differentiation state of the cell These discriminators are constructed from a learning model created by machine learning using image data of various cells and its staining data (correct image data).

The storage unit 31 is provided with a culture condition storage unit 312, a cell image storage unit 313, and an analysis result storage unit 314 in addition to the discriminator storage unit 311. The culture condition storage unit 312 stores information on the culture condition of the cell. In addition, the cell image storage unit 313 stores image data of a cell obtained by the microscopic observation unit 10 or another imaging device. Further, the analysis result storage unit 314 stores an analysis result including a measured value obtained by an analyzer other than the microscopic observation unit 10, such as a cell counter. Both the image data of the cell stored in the cell image storage unit 313 and the analysis result stored in the analysis result storage unit 314 are associated with the information on the culture condition stored in the culture condition storage unit 312.

Next, a procedure of cell analysis using the cell analysis system 1 of the present embodiment will be described.

First, a procedure of acquiring a cell image of the culture plate 13 including the cell 14 to be analyzed and analyzing the cell image will be described.

A user sets the culture plate 13 containing the cell 14 to be analyzed at a predetermined position of the microscopic observation unit 10, and performs a predetermined operation of instructing start of the cell image analysis by the input unit 51. In response to this, the microscopic observation unit 10 photographs a sample (the cell 14 in the culture plate 13), and executes phase calculation based on hologram data obtained by the photographing to generate phase image data.

When the phase image data is generated by the microscopic observation unit 10, the analysis result input receiving unit 33 displays a screen for inputting the culture condition and the like of the cell 14 on the display unit 52. In the present embodiment, project information (the type of the cell and the like), plate information (information on the culture conditions such as the type of culture medium and a total number of culture days), and image acquisition information (information such as the type and/or model number of the microscopic observation unit 10, a number of a well provided in the plate, and information on the number of culture days at the time of image acquisition) are input on this screen. The input information is stored as image identification information in the culture condition storage unit 312. The phase image data generated by the microscopic observation unit 10 is stored in the cell image storage unit 313 in association with the image identification information.

When the phase image data is stored in the cell image storage unit 313, the image analysis unit 32 displays a screen for allowing the user to select the analysis content on the display unit 52. The analysis content displayed here corresponds to the analysis content of the discriminator stored in advance in the discriminator storage unit 311, and includes, for example, determination of the number of cells, determination of the shape of the cell, determination of the cell coverage rate, and determination of the differentiation state of the cell, as described above.

When the user selects one or a plurality of analysis contents, the image analysis unit 32 reads out the type of the cell previously input and the discriminator corresponding to the selected analysis content from the discriminator storage unit 311, and analyzes the phase image data using the discriminator. The value (measured value) output from the discriminator is stored in the analysis result storage unit 314 in association with the image identification information.

The cell analysis system 1 of the present embodiment can also capture a cell image photographed by a microscope and the like other than the microscopic observation unit 10 connected to the cell analysis device 20. In this case, the user designates a storage location of the image data such as the phase image to read the image data, inputs the image identification information in the same manner as described above, and stores both the image data and the image identification information in the culture condition storage unit 312 and the cell image storage unit 313 in association with each other. In addition, the analysis content is selected in the same manner as described above, and a measured value is acquired using the discriminator corresponding to the analysis content, and is stored in the analysis result storage unit 314 in association with the image identification information.

In addition, in the cell analysis system 1 of the present embodiment, not only the measured value obtained by analyzing the cell image but also the analysis result such as the measured value acquired by analyzing the cultured cell by various analysis devices are managed.

When the user performs a predetermined input operation for instructing to import the analysis result such as the measured value acquired by an analyzer other than the cell analysis system 1 of the present embodiment, the analysis result input receiving unit 33 allows the user to designate the storage location of data of the analysis result to be imported. In addition, the screen for inputting the culture condition and the like of the cell 14 for which the analysis result has been acquired is displayed on the display unit 52. Also on this screen, the project information and the plate information are input in the same manner as described above. Here, analysis result acquisition information (information such as the type and/or model number of the analyzer in which the analysis result has been obtained, passage number, and the number of culture days at the time of analysis) is input instead of the image acquisition information. The input information is stored as analysis result identification information in the culture condition storage unit 312. In addition, the data of the analysis result stored in the location designated by the user is stored in the analysis result storage unit 314 in association with the analysis result identification information.

In the cell analysis system 1 of the present embodiment, by the above processing, the image data and the analysis result of the cell cultured under various conditions in various projects are stored in association with information such as the project and the culture condition. Thus, the data of the cell image obtained by the microscopic observation unit 10 and the like, the measured value obtained by analyzing the cell image, and the analysis results obtained by various analyzers other than the cell analysis system 1 can be centrally managed.

Next, a procedure related to the analysis of the measured value in the cell analysis system 1 of the present embodiment will be described.

When the user performs a predetermined input operation for instructing analysis of the measured value, the culture condition designation input receiving unit 34 displays, on the display unit 52, a screen for designating a target of analysis of the measured value from among the culture conditions stored in the culture condition storage unit 312.

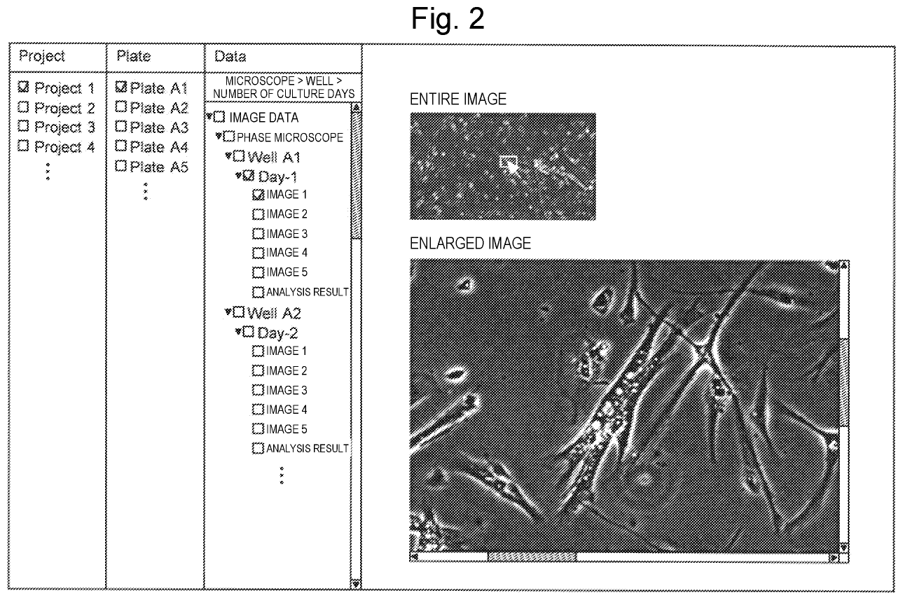

FIG. 2 is a display example of the screen, and a project selection field, a plate selection field, a data selection field, and an image display field are provided in order from the left side. In this example, when one project is selected in the project selection field, the plates cultured under the project are listed in the plate selection field. In addition, when one plate is selected in the plate selection field, the image data and analysis data obtained for the plate (plate cultured under a specific culture condition) are listed in the data selection field. When the user checks a box displayed together with the name of the image data, the corresponding image is displayed in the image display field. In the image display field, the entire image is displayed on an upper portion, and when the user selects a specific position in the entire image selects, an enlarged image of a selected position is displayed on a lower portion. The user can observe the image displayed in the image display field and consider whether to include the image in the target of analysis of the measured value.

When the user performs a predetermined input operation such as selecting one or a plurality of plates and pressing an analysis execution button, the correlation evaluation unit 35 reads out the plate information (information on the culture condition) of the selected plate from the culture condition storage unit 312. In addition, the analysis result associated with the culture condition read out from the culture condition storage unit 312 is read out from the analysis result storage unit 314. The read-out analysis result includes the measured values of a plurality of items. Hereinafter, a set of the measured values of the plurality of items is referred to as measured value set. In the present embodiment, there are a plurality of wells in one plate, and when one plate is selected, a plurality of the measured value sets are read out to culture the cell in each well.

Subsequently, the correlation evaluation unit 35 reads out the item of the measured value included in each of the plurality of measured value sets. Subsequently, the measured value set corresponding to two of the read-out items is read out from each plate, and scatter diagram data in which the measured value set is plotted on a two-dimensional plane with one item on the vertical axis and the other item on the horizontal axis is created. In addition, the correlation evaluation unit 35 creates an approximate straight line for each point of the created scatter diagram data, and obtains an error between the approximate straight line and each measurement point. Although the example of using the approximate straight line has been described here, it is possible to configure such that a quadratic function and an exponential function are set in advance in addition to such a linear function, and an approximate straight line or an approximate curve is created using the function having the smallest error among these functions.

The correlation evaluation unit 35 creates the scatter diagram data and the approximate straight line or the approximate curve in the same manner as described above for each of all combinations of two of the plurality of items read previously, and obtains an error. Then, the combinations of the two items are ranked in ascending order of error.

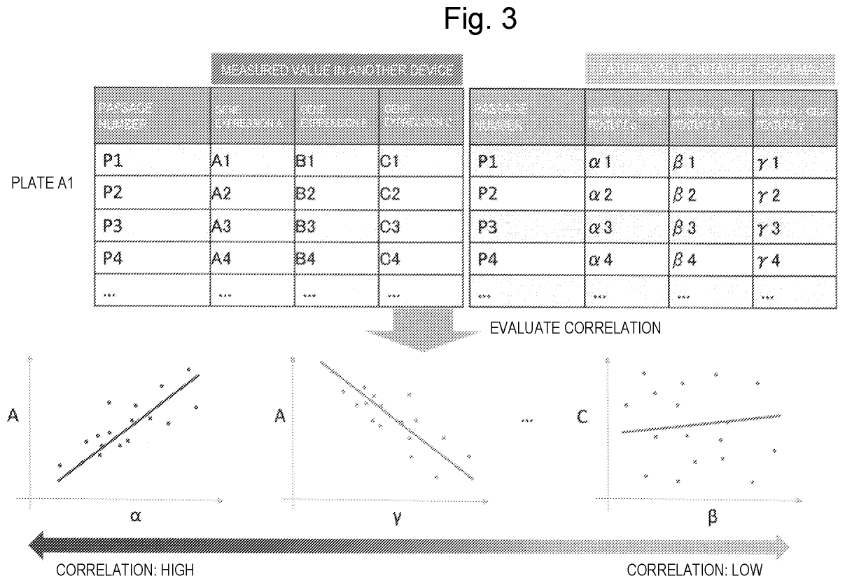

FIG. 3 shows an example of processing by the correlation evaluation unit 35. In this example, a plate A1 is selected. In this plate, cells having different passage numbers are cultured for each well, and for each, the measured values of items of morphological feature values α, β, and γ are obtained from the analysis of the cell image, and the measured values of items of gene expressions A, B, and C are obtained by another analyzer. That is, for one culture, there is the measured value set consisting of the measured values for six items. In this example, the correlation value evaluation unit 35 creates the scatter diagram data for all combinations (15 combinations of items) of two of the measured values of the six items, creates the approximate straight line or the approximate curve, and obtains an error to perform ranking.

When sets of items are ranked, the correlation item presentation unit 36 extracts a predetermined number of sets of items from the sets ranked higher, and displays the extracted sets of items on the screen of the display unit 52 together with a diagram in which the scatter diagram and the approximate straight line or the approximate curve are superimposed.

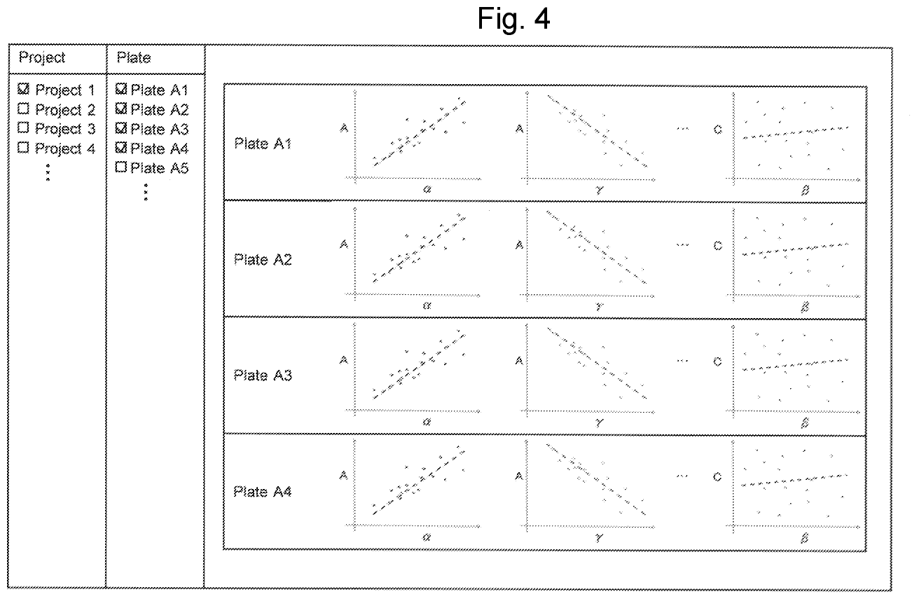

FIG. 4 is an example of screen display on the display unit 52 by the correlation item presentation unit 36 when four plates A1, A2, A3, and A4 (four culture conditions) are selected. In this example, for each plate, the scatter diagram and the approximate straight line are displayed from the left side in descending order of the combination having a high correlation of the measured values of the two items. The user can recognize the combination of the items having a high correlation of the measured value by viewing this display. In addition, the user can also confirm how the measured values of the two items are distributed.

In the cell analysis system 1 of the present embodiment, for example, as shown in FIG. 4, for each of the plurality of culture conditions, correlation between the measured value obtained from analysis of cell image data and the measured value obtained from analysis using another analyzer can be displayed. By getting a bird's-eye view of them, the user can easily confirm, for example, the combination of items having an extraordinarily high correlation of the measured value only under a specific culture condition and the combination of items having a high correlation of the measured value regardless of the culture condition.

The above-described embodiment is merely an example, and can be appropriately modified in accordance with the spirit of the invention. In the above embodiment, the example has been described in which the culture condition to be analyzed is designated by selecting the plate; however, the culture condition can also be designated by another method. For example, it is possible to configure such that a setting value of a culture parameter (for example, the type of culture medium) included in the culture condition is designated, and plates cultured using the culture medium of the type are collectively selected.

In the above embodiment, the scatter diagram and the approximate straight line are displayed from the left side in descending order of the combination having a high correlation of the measured values of the two items for each culture condition (plate); however, an appropriate mode can be adopted according to the purpose of analysis. For example, the correlation value evaluation unit 35 may extract such a combination of two items that the measured value of one item is unchanged with respect to a change in the measured value of the other item, and the correlation item presentation unit 36 may present the combination on the display unit 52. In addition, the measured values obtained for a plurality of designated culture conditions (plates) may be collectively processed. When cells are cultured under similar culture conditions, the correlation of the measured value is less likely to appear specifically only under one culture condition. In such a case, the number of sets of measured values can be increased by performing batch processing regardless of the culture condition, and accuracy of the approximate straight line and the approximate curve can be improved.

In the above embodiment, the cell analysis device 20 is combined with the microscopic observation unit 10 to constitute the cell analysis system 1; however, one or a plurality of other analyzers and the cell analysis device 20 may be combined to constitute the cell analysis system, or the cell analysis device 20 can be used alone.

In the cell analysis system 1 of the above embodiment, the cell analysis device 20 can be configured as a cloud server, and can be configured to be accessible from a plurality of research bases. By adopting such a configuration, it is possible to centrally manage data and analysis results of cell images obtained at the plurality of research bases in association with the culture conditions.

### Modes

It is understood by those skilled in the art that the plurality of exemplary embodiments described above are specific examples of the following modes.

### Clause 1

A cell analysis device according to one mode including:

a display unit;

a storage unit;

an analysis result input receiving unit configured to receive inputs of a plurality of culture conditions and measured values of a plurality of items obtained by analyzing a cell cultured under each of the plurality of culture conditions with a predetermined analyzer, and store the measured values in the storage unit associating the measured values of the plurality of items with a corresponding one of the plurality of culture conditions;

a culture condition designation input receiving unit configured to receive input of designation of some or all of the plurality of culture conditions;

a correlation evaluation unit configured to read out measured values of a plurality of items on the cell cultured under the designated culture condition from the storage unit, and evaluate a magnitude of correlation between the measured values of two items by a predetermined method for each of all combinations of two of the plurality of items; and

a correlation item presentation unit configured to display, on the display unit, a predetermined number of combinations extracted from among all the combinations based on an evaluation result by the correlation evaluation unit.

In the cell analysis device according to Clause 1, when a user inputs the measured values of the plurality of items obtained by analyzing a cell cultured under each of the plurality of the culture conditions with the predetermined analyzer together with the culture conditions of the cells, the analysis result input receiving unit associates the measured values with a corresponding one of the plurality of the culture conditions and stores them in the storage unit. Thereafter, when the user makes the input of designation of some or all of the plurality of culture conditions, the correlation evaluation unit reads out the measured values of the plurality of items on the cell cultured under the corresponding condition from the storage unit. Then, the magnitude of the correlation is evaluated for all combinations of two of the plurality of items by a predetermined method. The predetermined method is, for example, a method in which the correlation is evaluated larger as the difference between the measured values and an approximate straight line using a linear function or an approximate curve using a quadratic function is smaller. When the evaluation result for all combinations of items is obtained, the correlation item presentation unit extracts a predetermined number of combinations based on the evaluation result and displays the extracted combinations on the display unit. In the cell analysis device according to Clause 1, correlation between the measured values of two items may be easily observed only by looking at the items displayed on the display unit.

### Clause 2

In the cell analysis device according to Clause 1,

the analysis result input receiving unit is further configured to receive input of image data of the cell cultured under each of the plurality of culture conditions, and store the image data in the storage unit associating the image data for each culture condition.

In the cell analysis device according to Clause 2, for the cell cultured under each of a plurality of culture conditions, image data and the measured values of a plurality of items can be associated with each other and managed centrally.

### Clause 3

In the cell analysis device according to Clause 1 or Clause 2,

when the culture condition designation input receiving unit receives input designating a plurality of culture conditions,

the correlation item presentation unit is configured to extract the predetermined number of combinations individually for each of the plurality of culture conditions and display the result on the display unit.

In the cell analysis device according to Clause 3, it is possible to easily confirm the combination of items having an extraordinarily high correlation of the measured value only under a specific culture condition or the combination of items having a high correlation of the measured values regardless of the culture condition.

### Clause 4

In the cell analysis device according to any one of Clause 1 to Clause 3,

the correlation item presentation unit is further configured to display a scatter diagram of measured values on the display unit for the predetermined number of combinations.

In the cell analysis device according to Clause 4, a distribution state of the measured values can be confirmed on the display unit in addition to the magnitude of the correlation of the measured values.

### Clause 5

In the cell analysis device according to Clause 4,

the correlation item presentation unit is further configured to display the approximate straight line or the approximate curve representing the correlation between the measured values of two items on the scatter diagram.

In the cell analysis device according to Clause 5, it is possible to easily confirm deviation between each point and the approximate straight line or the approximate curve displayed on the scatter diagram, and observe distribution of the points.

### Clause 6

In the cell analysis device according to any one of Clause 1 to Clause 5,

the culture condition designation input receiving unit is configured to read out the culture condition stored in the storage unit, display the read-out culture condition on the display unit, and receive input of selection of the culture condition displayed on the display unit.

In the cell analysis device according to Clause 6, the culture condition to be analyzed can be easily designated without individually inputting a setting value of a culture parameter and the like included in the culture condition.

### REFERENCE SIGNS LIST

1 . . . Cell Analysis System

10 . . . Microscopic Observation Unit

11 . . . Light Source

12 . . . Image Sensor

13 . . . Culture Plate

14 . . . Cell

15 . . . Reference Light

16 . . . Object Light

20 . . . Cell Analysis Device

30 . . . Analysis/Processing Unit

31 . . . Storage Unit

311 . . . Discriminator Storage Unit

312 . . . Culture Condition Storage Unit

313 . . . Cell Image Storage Unit

314 . . . Analysis Result Storage Unit

32 . . . Image Analysis Unit

33 . . . Analysis Result Input Receiving Unit

34 . . . Culture Condition Designation Input Receiving Unit

35 . . . Correlation Evaluation Unit

36 . . . Correlation Item Presentation Unit

51 . . . Input Unit

52 . . . Display Unit

1. A cell analysis device comprising:
a display unit;
a storage unit;
an analysis result input receiving unit configured to receive inputs of a plurality of culture conditions and measured values of a plurality of items obtained by analyzing a cell cultured under each of the plurality of culture conditions with a predetermined analyzer, and store the measured values in the storage unit associating the measured values of the plurality of items with a corresponding one of the plurality of culture conditions;
a culture condition designation input receiving unit configured to receive input of designation of some or all of the plurality of culture conditions;
a correlation evaluation unit configured to read out measured values of a plurality of items on the cell cultured under the designated culture condition from the storage unit, and evaluate a magnitude of correlation between the measured values of two items by a predetermined method for each of all combinations of two of the plurality of items; and
a correlation item presentation unit configured to display, on the display unit, a predetermined number of combinations extracted from among all the combinations based on an evaluation result by the correlation evaluation unit.

2. The cell analysis device according to claim 1, wherein
the analysis result input receiving unit is further configured to receive input of image data of the cell cultured under each of the plurality of culture conditions, and store the image data in the storage unit associating the image data for each culture condition.

3. The cell analysis device according to claim 1, wherein
when the culture condition designation input receiving unit receives input of designation of a plurality of culture conditions,
the correlation item presentation unit is configured to extract the predetermined number of combinations individually for each of the plurality of culture conditions and display the result on the display unit.

4. The cell analysis device according to claim 1, wherein
the correlation item presentation unit is further configured to display a scatter diagram of the measured values on the display unit for the predetermined number of combinations.

5. The cell analysis device according to claim 4, wherein
the correlation item presentation unit is further configured to display an approximate straight line or an approximate curve representing the correlation between the measured values of two items on the scatter diagram.

6. The cell analysis device according to claim 1, wherein
the culture condition designation input receiving unit is configured to read out the culture condition stored in the storage unit, display the read-out culture condition on the display unit, and receive input of selection of the culture condition displayed on the display unit.
